(19) United States
(12) Patent Application Publication
(10) Pub. No.: US 2025/0302825 A1
(43) Pub. Date: Oct. 2, 2025

(54) COMBINATION TREATMENTS FOR DEPRESSION UTILIZING AN NMDAR ANTAGONIST

(71) Applicant: Glytech LLC, Dobbs Ferry, NY (US)

(72) Inventor: Daniel C. Javitt, Dobbs Ferry, NY (US)

(73) Assignee: Glytech LLC, Dobbs Ferry, NY (US)

(21) Appl. No.: 19/234,366

(22) Filed: Jun. 11, 2025

Related U.S. Application Data
(63) Continuation-in-part of application No. 17/844,087, filed on Jun. 20, 2022.
Application No. 17/844,087 is a continuation-in-part of application No. 16/166,101, filed on Oct. 21, 2018, now abandoned.
Application No. 16/166,101 is a continuation-in-part of application No. 15/650,912, filed on Jul. 16, 2017, now Pat. No. 10,660,887.
Application No. 15/650,912 is a continuation of application No. 13/936,198, filed on Jul. 7, 2013, now Pat. No. 9,737,531.
(63) Continuation-in-part of application No. 15/987,932, filed on May 24, 2018, now Pat. No. 10,583,138.
(63) Continuation-in-part of application No. 15/723,391, filed on Oct. 3, 2017, now abandoned.
Application No. 15/723,391 is a continuation-in-part of application No. 13/982,460, filed on Sep. 29, 2013, now Pat. No. 9,789,093.
(60) Provisional application No. 61/437,700, filed on Jan. 31, 2011.
(60) Provisional application No. 61/494,907, filed on Jun. 9, 2011.
(60) Provisional application No. 61/741,114, filed on Jul. 12, 2012.
(60) Provisional application No. 61/741,115, filed on Jul. 12, 2012.
(60) Provisional application No. 62/518,020, filed on Jun. 12, 2017.

(51) Int. Cl. A61K 31/496; A61K 31/06; A61K 31/135; A61K 31/138; A61K 31/381; A61K 31/42; A61K 31/431; A61K 31/4525; A61K 31/519; A61K 31/55; A61K 31/551; A61K 31/553; A61K 31/554; A61K 45/06

(52) U.S. Cl. CPC A61K 31/496; A61K 31/06; A61K 31/135; A61K 31/138; A61K 31/381; A61K 31/42; A61K 31/431; A61K 31/4525; A61K 31/519; A61K 31/55; A61K 31/551; A61K 31/553; A61K 31/554; A61K 45/06

(57) ABSTRACT

Provided herein are combination treatments for use in treatment of NMDAR antagonist-responsive disorders such as depression, and which can simultaneously alleviate the anxiogenic side effects of certain antidepressant and antipsychotic medications, thereby enabling continued and improved antidepressant and antipsychotic treatment. Methods for treatment of NMDAR-antagonist responsive disorders while simultaneously reducing medicament side effects, particularly anxiety, akathisia, and associated suicidality are also described herein.

### CROSS-REFERENCE TO RELATED APPLICATIONS

This is a Continuation-in-Part of U.S. patent application Ser. No. 17/844,087, filed Jun. 20, 2022, which is a Continuation of U.S. patent application Ser. No. 16/166,101, filed Oct. 21, 2018, which was a Continuation-in-Part of U.S. patent application Ser. No. 15/650,912, filed Jul. 16, 2017, now issued as U.S. Pat. No. 10,660,887, which is Continuation of U.S. patent application Ser. No. 13/936,198, filed Jul. 7, 2013, now issued as U.S. Pat. No. 9,737,531, which in turn claimed the benefit of the filing dates of U.S. Provisional Patent Application Nos. 61/741,114 and 61/741,115, both of which were filed on Jul. 12, 2012. This application also is a Continuation-in-Part of U.S. patent application Ser. No. 15/987,932, filed May 24, 2018, now issued as U.S. Pat. No. 10,583,138, which claims the benefit of the filing date of U.S. Provisional Patent Application No. 62/518,020, filed Jun. 12, 2017. This application is further a Continuation-in-Part of U.S. patent application Ser. No. 15/723,391, filed Oct. 3, 2017, which is the Continuation of U.S. patent application Ser. No. 13/982,460, filed Sep. 29, 2013, now issued as U.S. Pat. No. 9,789,093, which was the US National Stage of International Patent Application No. PCT/IL2012/050034, filed Jan. 30, 2012, and which in turned claimed the benefit of the filing date of US Provisional Patent Application Nos. 61/437,700, filed Jan. 31, 2011, and 61/494,907, filed Jun. 9, 2011. The contents of the foregoing patent applications are incorporated by reference herein in their entireties.

### FIELD

This application relates to combination compositions for use in treatment of NMDAR-antagonist responsive disorders such as depression, anxiety disorders, pain, OCD and PTSD and which can simultaneously alleviate the anxiogenic side effects of certain antidepressant and antipsychotic medications, thereby enabling continued and improved antidepressant and antipsychotic treatment. Methods for treatment of depression while simultaneously reducing medicament side effects, particularly anxiety, akathisia, and associated suicidality are also described herein.

### BACKGROUND

In recent years, several neuropsychiatric conditions including depression (e.g., major depression and bipolar depression), obsessive compulsive disorder (OCD), post traumatic stress disorder (PTSD) and pain conditions have been shown to be responsive to antagonists of N-methyl-D-aspartate type glutamate receptors (NMDAR). Of these, depression is among the most common in the adult population.

Major depression is a clinical syndrome that includes a persistent sad mood or loss of interest in activities, which persists for at least two weeks in the absence of treatment. Symptoms of major depression are typically measured using rating scales such as the Hamilton Depression Rating Scale (HAM-D) or the Beck Depression Inventory (BDI). In addition to including symptoms relevant to depressed mood, the HAM-D also contains symptoms sensitive to psychosis, including items for guilt, depersonalization/derealization and paranoia.

Major depression may also be associated with symptoms of anxiety, which may be measured with rating scales such as the Hamilton Rating Scale for Anxiety (HAM-A). Depressive disorders are divided in major depression (MDD) and bipolar depression (BPD). Major depression may also occur with and without melancholic features. In addition, depressive symptoms may occur in the context of anxiety disorders such as generalized anxiety disorder, dissociative disorders, personality disorders or adjustment disorders with depressed mood (DSM-IV).

Depression may also be accompanied by psychotic features such as hallucinations or delusions. Psychotic depression can occur in the context of either MDD or BPD. In the case of psychotic depression, use of antipsychotic agents along with antidepression agents may be beneficial.

Current treatments for major depression consist primarily of older antidepressants, such as monoamine oxidase inhibitors (MAOI) and tricyclic antidepressants (TCAs) that were first developed in the 1960's, and newer agents such as phenylpiperazine antidepressants, tetracyclic antidepressants (TeCAs), selective serotonin (SSRI), serotonin/norephinephrine (SNRI) reuptake inhibitors and norepinephrine-dopamine reuptake inhibitors (NDRI). TeCAs have also been termed noradrenergic and specific serotonergic antidepressants (NaSSAs). MAOIs and TCAs are considered “broader spectrum” agents than SSRIs/SNRIs that were developed subsequently.

Despite their efficacy in many cases, current approaches have severe limitations. Only 60-65% of patients respond to the initial regimen and among those responding, less than half either reach remission or become symptom-free. Individuals not responding to a first course of antidepressant treatment are often switched to a different drug, with results that are generally modest and incremental. Combinations of antidepressants have not been shown to be superior to monotherapy for refractory depression and typically increase risk of side effects, and so are not recommended. Thus, a continuing need exists for additional treatments for depression and particularly major depression that are both more broadly effective and result in reduced treatment-associated side effects.

### SUMMARY OF THE INVENTION

This invention is directed at the combination of ketamine agents with specific antidepression agents for the treatment of depressive disorders with reduced side effects relative to the use of ketamine alone. Ketamine agents may include racemic ketamine (R,S)-ketamine, S-ketamine (esketamine) or R-ketamine (arketamine).

The foregoing and other objects, features, and advantages will become more apparent from the following detailed description, which proceeds with reference to the accompanying figures.

### BRIEF DESCRIPTION OF THE DRAWINGS

FIG. 1: Average locomotor activity following injection of the indicated compounds over 30 min prior to amphetamine administration. Locomotor activity is measured as distance traveled (cm) per 5 min. Bars represent the marginal mean values+/−standard error of the mean derived from an ANOVA across all indicated conditions. The ketamine data are collapsed across the different ketamine agents. The dashed line shows mean locomotor activity in the presence of ketamine alone (combined agents). #p<0.05 ketamine alone vs. control in the no antidepressant condition (post-hoc LSD). *p<0.05, ***p<0.001 difference in locomotor activity in the presence of combined antidepressant+ketamine vs. ketamine alone for the indicated conditions.

FIG. 2: Average locomotor activity following injection of the indicated compounds over the 60 min following amphetamine injection. ##p<0.01: ketamine alone vs. control in the no antidepressant condition. *p<0.05: vs. no antidepressant; ***p<0.001: difference in locomotor activity in the presence of combined antidepressant+ketamine vs. ketamine alone for the indicated conditions. The dashed line indicates mean locomotor activity in the ketamine alone condition during the pre-amphetamine administration period, as shown in FIG. 1.

FIG. 3: Locomotor activity in the presence of higher dose (100 mg/kg) ketamine. *p<0.05, ***p<0.001 difference between the indicated conditions.

FIG. 4: Open arm entries during the elevated plus maze (EPM) in the indicated conditions. Values are estimated marginal means+/−sem for the indicated conditions from an ANOVA across all conditions. *p<0.05, ***p<0.001 between the indicated conditions. NS: not significant.

FIG. 5: Time inactive in the forced swim test (FST) under the indicated conditions. ***p<0.001 between the indicated conditions.

### DETAILED DESCRIPTION OF THE INVENTION

### Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Similarly, use of the word “about” in front of a numerical value or range indicates that values on either side, typically ±10%, of the stated value or range can be considered to fall within the scope of that value or range. Although methods and materials similar or equivalent to those described herein can be used in the practice or testing of this disclosure, suitable methods and materials are described below. The term “comprises” means “includes.” “Consisting essentially of” indicates a composition, method, or process that includes only those listed features as the active or essential elements, but can include non-active elements in addition. Additionally, where used herein, “comprises” encompasses the more restrictive embodiments of “consisting essentially of” and “consisting of.” The abbreviation, “e.g.” is derived from the Latin exempli gratia, and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

Antidepression agent: As used herein, an “Antidepression Agent” can be a compound commonly recognized as an antidepressant agent or can be a compound currently recognized as an antipsychotic agent, but which has also been shown to have antidepressant effects.

Current treatments for major depression consist primarily of older antidepressants, such as monoamine oxidase inhibitors (MAOI) and tricyclic antidepressants (TCAs) that were first developed in the 1960's, and newer agents such as phenylpiperazine antidepressants, tetracyclic antidepressants (TeCAs), selective serotonin (SSRI), serotonin/norephinephrine (SNRI) reuptake inhibitors and norepinephrine-dopamine reuptake inhibitors (NDRI). Currently marketed MAOIs include phenelzine, tranylcypromine, selegeline, and isocarboxazid. Currently marketed TCAs include amitryptiline, nortriptyline, desipramine, doxepin, norpramin, amoxapine, clomipramine, imipramine, protriptyline, and trimipramine. Currently marketed phenylpiperazine antidepressants include trazodone and nefazodone. Currently marketed SSRIs include sertraline, escitalopram, citalopram, fluoxetine, paroxetine, and fluvoxamine. Currently marketed SNRIs include venlafaxine, duloxetine, desvenlafaxine, milnacipran, and levomilnacipran. Currently marketed NDRIs include bupropion, methylphenidate and nomifensine. Other miscellaneous antidepressants include vortioxetine, vilazodone, 5-hydroxytrytophan and St. John's wort, zuranolone, gepirone and brexanolone. Currently marketed TeCAs include mianserin and mirtazapine. Mirtazapine is a racemic mix of S-mirtzapine and R-mirtazapine, which may have differential effects when administered individually relative to the racemic. Antidepression drugs may also be formulated for sustained release. Examples include Effexor XR (venlafaxine) or Drizalma Sprinkle (duloxetine). Approved dosing levels for these treatments may be found using standard references such as the prescribers' Desk Reference (PDR), which may also be found online at pdr.net. The information is also included in the package insert for each of the agents, which is approved by the US Food and Drug Administration (FDA), as well as online at the US FDA (accessdata.fda.gov/scripts/cder/daf/index.cfm). For example, duloxetine may be used at doses of 20-120 mg/day, venlafaxine may be used at doses of 75-150 mg/day. Doses are optimally adjusted to achieve the maximal benefit for each individual. Dosages outside of the recommended range may also be used at physician discretion.

TCAs and SSRIs show approximately equal efficacy in treatment of non-melancholic forms of depression, suggesting overlapping but differentiable mechanisms of action. TCAs as a group show limited antipsychotic activity, alone or in combination with antipsychotics, but may be effective in treating persistent depressive symptoms in stabilized schizophrenia patients. TCAs have been shown to worsen psychosis in acutely decompensated schizophrenia patients, but to be relatively without effect on psychosis during the chronic phase of illness. In contrast, SSRIs and TeCAs may improve psychotic symptoms in addition to treatment of depression in refractory schizophrenia, suggesting a differential mechanism of action and mild antipsychotic potency.

Antipsychotic agents may be divided into typical (e.g. chlorpromazine, haloperidol) vs. atypical (e.g. risperidone, olanzapine, quetiapine, aripiprazole, clozapine, lurasidone, cariprazine) based upon receptor binding, preclinical effects and side effect profile. Several antipsychotic agents are also indicated for treatment of depression in both major depressive and bipolar disorders. Potentially beneficial antipsychotic medications include but are not limited to risperidone, olanzapine, quetiapine, quetiapine XR, aripiprazole, clozapine, iloperidone, sertindole, asenapine, lurasidone, cariprazine and brexpiprazole. The atypical antipsychotic roluperidone (also known as MIN-101) is under clinical investigation for the treatment of schizophrenia at doses of 32-64 mg/day.

Antidepression agents may increase liability for akathisia. Akathisia in turn is associated with reduced quality of life and mental health (Akgoz et al., Evaluation of akathisia in patients receiving selective serotonin reuptake inhibitors/serotonin and noradrenaline reuptake inhibitors. Behavioural Pharmacology. 2024; 35:460-3). Akathisia may also be associated with increased risk for suicidality (Hansen L. A critical review of akathisia, and its possible association with suicidal behaviour. Hum Psychopharmacol 2001; 16:495-505). All current medications for depression include a black-box warning regarding their liability to increase suicide risk.

Effective amount or a Therapeutically effective amount of therapeutic agents is meant to indicate a nontoxic but sufficient amount of the same to provide the desired effect. In a combination therapy of the present invention, an “effective amount” of one component of the combination is the amount of that compound that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation. The active agents described herein (e.g., D-methadone and the antidepression agents) are provide in therapeutically effective amounts known to the art.

Major Depression, Depression (generally): a clinical syndrome that includes a persistent sad mood or loss of interest in activities, which persists for at least two weeks in the absence of treatment. Symptoms of major depression are typically measured using rating scales such as the Hamilton Depression Rating Scale (HAM-D) or the Beck Depression Inventory (BDI). In addition to including symptoms relevant to depressed mood, the HAM-D also contains symptoms sensitive to psychosis, including items for guilt, depersonalization/derealization and paranoia. Other forms of depression include atypical depression, agitated depression, depression with mixed emotional features, cyclothymia, dysthymia minor depression and adjustment disorder with depressed mood. Bipolar depression may be divided into Bipolar I and Bipolar II subtypes based upon presence or absence of manic episodes. In bipolar disorder, depressive symptoms can occur in the context of either a depressive episode, or a mixed state in which symptoms of mania and depression occur simultaneously or in rapid sequence. Rapid cycling between mania and depressive episodes may also occur in some individuals.

As described in Textbook of INTERNAL MEDICINE, Kelley, et al. (eds.), Part X: Neurology, Chapter 469: Major Psychiatric Disorders, (J. Lippincott Co., Philadelphia), pp. 2198-2199 (1992), depression can occur throughout life and is at least twice as common in women as in men. Patients often present without the subjective sense of being depressed but complaining of somatic symptoms of depression, most commonly fatigue, sleep disturbances, or impotence. Patients may describe feeling sad, blue, low, irritable, or anxious, as well as being depressed. Diagnosis of major depression is based either on a distinct change of mood that is prominent, generally persists throughout the day, and occurs each day for at least 2 weeks or on markedly diminished interest or pleasure in most activities over a similar period. The diagnosis requires that at least four of the following symptoms be present nearly every day for a period of 2 weeks: significant weight loss (or weight gain in some younger patients), prominent sleep disturbance, agitation or retardation with slow speech, fatigue, feelings of worthlessness and guilt, slowed thinking, and hopelessness. Depression can likewise be associated with the symptoms of disease (e.g., systemic lupus erythematosus) or as a side effect of the treatment of disease (e.g., with antihypertensive therapy). One form of depression, postpartum depression, has been commonly found in women during the period following childbirth.

Treating and Treatment refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual.

Treatment-refractory depression (TRD) refers to a form of depression that responds poorly to currently available treatments (e.g., see nimh.nih.gov/trials/practical/stard/index.shtml June 2011) and which may have different underlying etiopathological mechanisms compared with other forms of depression. Combinations of antidepressants have not been shown to be superior to monotherapy for refractory depression and typically increase risk of side effects and are not recommended. Currently approved treatments for TRD include electroconvulsive therapy (ECT), transcranial magnetic stimulation (TMS) and intranasal s-ketamine.

D-cycloserine, or DCS, refers to the chemical D-cycloserine (CA Index Name: 3-Isoxazolidinone, 4-amino-, (4R)-(9CI); CAS Registry No. 68-41-7), or pharmaceutically acceptable salts thereof. DCS is an FDA (United States Food and Drug Administration)-approved drug for treatment of tuberculosis, and is sold by Eli Lilly and Company under the trade name Seromycin®. DCS is a structural analog of D-alanine, and is a broad-spectrum antibiotic produced by some strains of Streptomyces orchidaceus and S. garphalus.

### Compositions and Methods for Treatment of Depression, Chronic Pain and Other NMDAR Related Neuropsychiatric Disorders

Described herein is a formulation that comprises, consists essentially of, or consists of two components for the treatment of depression, chronic pain and potentially other NMDAR related neuropsychiatric disorders including obsessive compulsive disorder (OCD), post-traumatic stress disorder (PTSD), pain and anxiety disorders.

The first of these components includes an NMDAR antagonist The second of these components is an antidepression agent.

In a preferred embodiment, the NMDAR antagonists consists of ketamine ((R.S)-2-(2-Chlorphenyl)-2-(methylamino)cyclohexan-1-on), S-ketamine (S-2-(2-Chlorphenyl)-2-(methylamino)cyclohexan-1-on) or R-ketamine (R-2-(2-Chlorphenyl)-2-(methylamino)cyclohexan-1-on). In particular embodiments, the antidepression agent is a compound commonly known as an antidepressant, such as but not limited to those drawn from a list that includes MAOI, TCAs, phenylpiperazine antidepressants, TeCAs, SSRIs, SNRIs, and NDRIs.

Particular antidepressants for use in the described compositions and methods include the SSRIs citalopram provided at a dose of 20-60 mg/day, sertraline provided at a dose of 50-200 mg/day or fluoxetine provided at a dose of 20-80 mg/d; the SNRIs venlafaxine provided at a dose of 75-150 mg/day, duloxetine provided at a dose of 20-120 mg/day, milnacipran provided at a dose of 50-200 mg/d or levomilnacipran provided at a dose of 20-120 mg/d; the TeCA mirtazapine, provided at a dose of 15-45 mg/d; or the NDRI bupropion provided at a dose of 100-450 mg/day

In other embodiments, the antidepression agent is a compound commonly known as an antipsychotic (used synonymously herein with “antipsychotic agent”). The antipsychotic can be a typical or atypical antipsychotic, which can in some embodiments be a serotonin dopamine antagonist (SDA). In some embodiments, the antipsychotic agent is selected from the group consisting of amisulpride, aripiprazole, asenapine, bioanserin, bifeprunox, brexpiprazole, cariprazine, clotiapine, clozapine, iloperidone, lumateperone, lurasidone, mosaproamine, olanzapine, paliperidone, perospirone, quetiapine, remoxipride, risperidone, sertindole, sulpiride, ziprasidone, zotepine.

Particular atypical antipsychotic agents include risperidone, provided at a dose of 2-16 mg/day, lurasidone provided at a dose of 40-160 mg/d; brexpiprazole provided at a dose of 0.5-4 mg/d, olanzapine provided at a dose of 5-40 mg/d and roluperidone provided at a dose of 16-128 mg/day

Also described are treatments using the above pharmaceutical formulation for treatment of depressive disorders, which may occur in the context of major depressive disorder, bipolar disorder, anxiety disorders, obsessive compulsive disorder (OCD), or post traumatic stress disorder (PTSD). Risk for suicide is significantly increased in depressive disorders, but may respond differentially to medication versus depressive symptoms as a whole. When suicide occurs, it is often accompanied by feelings of worthlessness or inappropriate guilt, as well as recurrent thoughts of death or suicidal ideation and guilt is an accepted proxy for suicide. While the risk of suicide increases in subjects with a depressive disorder, medications used to date to typically treat depressive disorders paradoxically increase suicidal tendencies. The compositions and methods described herein, by simultaneously treating depressive and suicide-related psychomimetic symptoms thus simultaneously treat depression and associated suicidal ideation that frequently accompanies depression.

### The Relationship Between NMDAR and Neuropsychiatric Conditions

Most current theories of depression focus on serotonergic and/or noradrenergic brain systems. Glutamate is an alternative brain neurotransmitter that has been studied to a limited degree in relationship to depression or other affective disorders. Glutamate binds to several receptor types including N-methyl-D-aspartate type glutamate receptors (NMDAR). NMDAR contain multiple binding sites including an agonist site for glutamate and an allosteric modulatory site (aka glycineB receptor, strychnine-insensitive glycine receptor) sensitive to the endogenous brain amino acids glycine and D-serine. Agonists at the glycine site increase NMDAR activation in response to glutamate while antagonists decrease NMDAR activation.

Functional agonists and antagonists at the glycine site can be identified using well-validated electrophysiological assays such as modulation of NMDA-receptor mediated responses to NMDA glutamate-site agonists, or radioreceptor assays, such as modulation of binding to the NMDA PCP-receptor channel binding site. Glycine site agonists and antagonists can also be distinguished based upon both electrophysiology and receptor binding from compounds such as phencyclidine (PCP) or ketamine that bind to the channel site (aka PCP receptor, uncompetitive antagonist site) of the NMDAR. Effective agonists and antagonists may be identified, for example, as compounds with <100 nM affinity for their target and >10-fold selectivity vs. other relevant targets. Partial agonists are defined as compounds that have reduced efficacy for inducing conformational change in receptors (typically 40-80%) relative to full agonists, and which may induce agonist effects at low dose but antagonist effects at high dose.

Relatively few studies have investigated glutamate- or NMDAR-related measures in depression. To the extent that it has been studied, it has been suggested that depression is associated with reduced NMDAR function (e.g. Frye et al., 2007). Sumiyoshi et al. (2004) found to no difference in plasma glycine levels relative to controls. In contrast, other studies have linked high glycine levels to poor response to SSRIs, possibly in association with abnormalities in the glycine hydroxylase (decarboxylating) (aka decarboxylase, GLDC) gene suggesting that treatment refractory depression may constitute an etiologically distinct form of the disorder (Ji et al., 2011). Depression may be modeled in rodents using assays such as learned helplessness, forced swim or tail suspension tests. There are at present no animal models specifically sensitive to treatment refractory depression. Elevated glycine levels are also reported in relapsing mania. For example, Hoekstra et al., 2006 reported mean plasma levels of 283.3±102.7 for manic patients vs. a mean of 224.0±51.5 for controls (p=0.02).

Recently, the non-competitive NMDAR antagonist ketamine has also been shown to have antidepressant effects in humans when tested in individuals with treatment-resistant depression. The compound shows similar effects in both unipolar and bipolar depression. Other non-competitive NMDAR antagonists such as MK-801 also show anti-depressant effects in animal models. However, antidepressant effects induced by ketamine are associated with exacerbation of psychosis, which greatly reduces their utility in clinical situations. Currently approved NMDAR antagonists for the treatment of depression include intranasal esketamine and combined dextromethorphan and bupropion.

A double-blind, randomized, placebo-controlled clinical trial evaluating the NMDAR-2B subunit-selective antagonist CP-101,606 found that this agent also induced significant and relatively rapid antidepressant effects in patients with treatment-resistant MDD. As with ketamine, however, CP-101,606 was used by intermittent IV infusion, limiting its clinical utility. Moreover, as with ketamine, significant dissociative effects emerged during CP-101,6060 infusion. However, other NMDAR antagonists, such as the extrasynaptic NMDAR antagonist memantine, have not shown beneficial clinical results for the treatment of depression, suggesting that blockade of intrasynaptic receptors may be critical

### D-Cycloserine as an NMDAR Antagonist is Effective in Treating NMDAR Responsive Conditions

D-cycloserine is a compound currently approved for treatment of tuberculosis (TB). Psychotropic effects of D-cycloserine were noted in the late 1950's in patients being treated for TB. In an initial report, effects of D-cycloserine were noted on symptoms such as anorexia, asthenia and insominia. However, no formal psychiatric diagnoses were made. Furthermore, cycloserine was recommended primarily for treatments of tension and insomnia, as opposed to depression.

Formal further studies with D-cycloserine were not pursued until the 1980's when it was observed that D-cycloserine functions as a partial agonist (mixed agonist/antagonist) at the glycine binding site of the NMDAR, with agonist effects predominating at low dose and antagonist effects predominating at high dose. As compared to glycine, D-cycloserine shows approximately 50% efficacy in stimulating NMDA receptors when used at maximal concentration.

Because of its ability to bind to NMDAR and because of theories linking NMDAR to schizophrenia, D-cycloserine has been studied in treatment resistant schizophrenia. At low doses, D-cycloserine has been found to produce beneficial effects in some but not all studies, and may exacerbate symptoms in individuals receiving clozapine. Furthermore, at higher doses (>250 mg), however, D-cycloserine exacerbates psychosis and so according to package label insert is contra-indicated in schizophrenia, depression and anxiety disorders.

D-cycloserine has also been assessed in the treatment of anxiety disorders, PTSD and enhancement of learning and memory at doses of 50-500 mg, with the goal primarily of enhancing NMDAR function. In addition, use of D-cycloserine has been claimed for enhancement of cognition at doses of up to 100 mg and for treatment of a wide variety of neuropsychiatric disorders at doses of up to 500 mg. It has also been taught that D-cycloserine may be useful in augmenting cognition in Parkinsons disease.

In both anxiety and schizophrenia studies, it was also noted that effects of D-cycloserine may decrease over time during repeated treatment, leading some to advocate use of weekly, rather than daily, D-cycloserine. When used as augmentation of behavior therapy for anxiety, D-cycloserine is commonly used episodically in combination with behavioral therapy sessions.

Initial research with D-cycloserine in preclinical models did not suggest its usefulness at high dose in treatment of depression. Partial agonists of NMDAR, in particular 1-aminocyclopropanecarboxylic acid (ACPC), were reported to have efficacy in animal models, but were not tested in human studies. When used in these models, D-cycloserine was noted to have inconsistent effects and to be less effective than either ACPC or imipramine. Furthermore, effects were only observed at the lowest dose tested, arguing away from high dose treatment in humans. In animal depression models, tolerance over weeks was observed, arguing against sustained long-term use. D-cycloserine reported for use at a dose of 250 mg/day was found to be without significant effect on symptoms of major depression and moreover, commonly available prescribing information states that D-cycloserine use is contraindicated in individuals with a history of epilepsy, depression, severe anxiety, or psychosis (Lilly. Seromycin (cycloserine) capsules prescribing information. Indianapolis, Ind.; 2005 Apr. 28). However, subsequent studies with higher doses of D-cycloserine have documented its effectiveness in treating depression in both human and animal models, as described in U.S. Pat. Nos. 9,789,093 and 10,583,138, and below

Human Studies with D-Cycloserine

In humans, clinical studies use doses of 500-2000 mg/day, which produce plasma levels between 25-125 microgram/mL. For example, in one study individuals with treatment resistant depression were treated with escalating doses of D-cycloserine over a period of 6 weeks. D-cycloserine was added to their existing antidepression treatment. The patients had been receiving a stable therapeutic dose of an approved antidepressant drug for at least 8 weeks before study entry. All patients met criteria for refractory depression, defined as a >20 score on the 21-item HAM-D, despite at least 2 prior adequate antidepressant medications (e.g. SSRIs, SNRIs, TCAs) trials during the current depression episode. Ongoing medication doses remained fixed throughout the study.

After complete description of the study, orally and in writing, written, informed consent was obtained from all participating patients. A random assignment, double-blind placebo-controlled, parallel group design was used in the study. After a 2 wk (week −2 to baseline) assessment period, subjects were randomly allocated to receive under double blind conditions either D-cycloserine or placebo for 6 wk. D-cycloserine or placebo were given in addition to each patient's regular antidepressant medication, the dose of which remained fixed throughout the study. D-cycloserine and placebo were administrated orally, in identical capsules, and according to the same dose escalation schedule. Plasma glycine and serine levels were determined pre/post treatment for a subgroup of 20 subjects by HPLC.

A fixed, slow titration-high dose treatment schedule for adjuvant treatment with D-cycloserine was conceptualized and used with all participating patients during the 6 wk study period: 250 mg/day for 3 days ⋄ 500 mg/day for 18 days ⋄ 750 mg/day for one week ⋄ 1000 mg/day (1 g/day) for two weeks (see U.S. Pat. No. 9,789,093).

Several scales were used throughout the study to assess the severity of symptoms and side effects in each patient. All the assessments were performed by a psychiatrist who was blind to the experimental treatment assignment. HAM-D was used at wk. −2, baseline and bi-weekly throughout the study. HAM-A, BDI and the Clinical Global Impression—Severity of Illness Scale were used at baseline and biweekly throughout the study. In addition to the symptoms sensitive to psychosis development that are included in HAM-D, overall side effects were assessed at baseline and biweekly throughout the study using the UKU side effects rating scale for the registration of unwanted effects of psychotropic drugs. BDI scores were available only for 20 of the 26 study participants.

Primary data analysis was conducted by mixed-model regression using all available data. Subject id was coded as index variable, treatment week (0-6) as repeated measure, and treatment as fixed factor. A secondary analysis assessed effect size of change scores from baseline to end of treatment based upon LOCF measures for all subjects. Significance of change across treatment week was evaluated using repeated measures ANOVA. Follow-up t-tests were performed to evaluate differential response by older (TCA) vs. newer (TeCA, SSRI/SSRI) treatment.

Thirteen patients were randomly allocated to receive adjuvant D-cycloserine treatment and thirteen patients were randomly allocated to receive adjuvant placebo. A total of twenty-two patients, ten in the D-cycloserine group and twelve in the placebo group completed the entire study. Four patients were withdrawn from the study: one in the placebo group, due to complaints of chest pain, and three in the D-cycloserine group due to non-compliance, and complaints of ear aches and tiredness, respectively. Following withdrawal from the study, these complaints ceased. No other complaints were registered throughout the study and no D-cycloserine/placebo treatment side effects were noted using the UKU scale for rating side effects.

As reported in Heresco-Levy et al., Int. J Neuropsychopharmacol, 3:501-6, 2013, D-cycloserine treatment led to significant improvement in depressive symptoms as measured by HAM-D (p=0.005) and the BDI (p=0.046). When HAM-D change scores were analyzed as a function of pretreatment glycine levels, including glycine <300 μM vs. ≥300 μM as a factor led to a significant treatment X glycine level interaction (p=0.043). Effect size vs. placebo among patients with pretreatment glycine ≥300 μM (n=14) was extremely large (d=2.36), suggesting robust antidepressant effects. Similarly, 4 of 7 (57%) patients with pre-treatment glycine levels ≥300 μM were remitters vs. only 1 of 5 (20%) of non-remitters, suggesting that patients with elevated pretreatment glycine levels show unexpected and particular sensitivity to glycine antagonist treatment.

Trends toward improvement were observed as well for anxiety, as reflected in the HAM-A (p=0.051) and overall level of function, as reflected in the CGI-S (p=0.076). No significant change was observed in items potentially reflecting psychosis, including depersonalization/derealization and paranoia, which remained negligible in both treatment groups throughout the study.

Only one subject in the study (D-cycloserine group) had significant suicidal ideation on study entry, as reflected in HAM-D suicide item (item 3)>2. In this subject, symptoms resolved within two weeks and remained reduced throughout the remainder of the study. No significant effect on guilt feelings was observed in the primary analysis. Nevertheless, a significantly greater to (p=0.01) reduction in guilt was observed in a completers analysis, with patients on D-cycloserine (n=10) showing a 1.3±0.5 pt reduction in guilt feelings vs. 0.4±0.9 in patients on placebo (n=11), also suggesting reduced suicide risk.

Comparison by antidepressant type: Of the 13 patients randomized to active treatment, 11 were being treated with newer antidepressants including SSRIs/SNRIs or TeCAs, and 2 with TCAs. Comparison of response between patients receiving older vs. newer antidepressants showed a significantly higher response and remission rate among those receiving newer antidepressants, as well as a between-group difference in depressive symptoms at week 21 using the HAM-D. In addition, both of the patients receiving TCAs (100%) showed non-zero psychosis scores at week 6, vs. 5 of 10 receiving newer antidepressants (50%). However, no subjects were discontinued from either group because of psychotic symptoms.

In a subsequent study, 22 individuals with bipolar disorder were randomized to receive combined D-cycloserine and lurasidone vs. lurasidone alone following IV ketamine treatment. D-cycloserine was administered at doses of 500-1000 mg/day. The combination of D-cycloserine and lurasidone was significantly more effective in maintaining improvement in depression and reducing suicidal depression than lurasidone alone (Nierenberg et al., Int J Bipolar Disord 2023; 11, 28). In a subsequent study, D-cycloserine treatment reduced akathisia induced by lurasidone treatment.

Animal Studies with D-Cycloserine

In animal models, D-cycloserine was found to significantly reduce immobility time in the rodent forced swim test at doses in excess of 100 mg/kg. These doses were associated with sustained blood plasma concentrations in excess of 25 microgram/mL but lower than 125 microgram/mL. The anti-depression effects of D-cycloserine remained significant when given in combination with an anti-depression agent such as lurasidone (see e.g., U.S. Pat. No. 10,583,138).

The anti-depression effects of D-cycloserine are also additive to those produced by other anti-depressant types, including TCAs, SSRIs, SNRIs, and others (e.g. buproprion, mirtazapine) (e.g. US 2022/0143041A1), suggesting unexpected benefit from the combination. In general, use of agents with convergent therapeutic effects but differential side effects will maximize therapeutic efficacy while reducing side effects.

Animal studies have also shown unexpected synergies between D-cycloserine and anti-depression agents. For example, as described above, anti-depression agents may increase the liability for anxiety and akathisia in humans. These symptoms can be modeled using the rodent elevated plus maze (EPM) test, which assesses the degree to which animals remain in the open versus closed arm of the apparatus. In general, reduced time in the open arm in rodents is considered a model of increased liability for producing anxiety or akathisia in humans. In rodents, atypical antidepressants including lurasidone, cariprazine, risperidone and quetiapine decreased time in the open arm of the EPM, suggestive of increased liability to produce anxiety and/or akathisia. These effects were reversed by simultaneous treatment with the glycine-site antagonist D-cycloserine, but not the non-competitive antagonist ketamine (e.g. U.S. Pat. No. 10,583,138).

NMDAR antagonists may also produce psychotomimetic effects in humans that can be modeled in rodents using a locomotor activity (LMA) assay. For example, D-cycloserine at doses relevant to its antidepression effects (300-1000 mg/kg) increases rodent locomotor activity. The effects of D-cycloserine were reversed by anti-depression agents, especially those with low serotonergic transport inhibition relative to other targets such as norepinephrine transporters (e.g. U.S. Pat. No. 10,881,665).

### Therapeutic Efficacy of Additional NMDAR Antagonists

In addition to D-cycloserine, other NMDAR antagonists have also been described that may be useful in the treatment of depression. Non-limiting examples of NMDAR antagonists for use in the described compositions and methods include ketamine, Selfotel, aptiganel, CPP, CGP-37849, felbamate, Gavestinel N-(6,7-dichloro-2,3-dioxo-1,2,3,4-tetrahydro-quinoxalin-5-yl)-N-(2-hydroxy-ethyl)-methanesulfonamide and 6,7-dichloro-5-[3-methoxymethyl-5-(1-oxypyridin-3-yl)-[1,2,4]triazol-4-yl]-1,4-dihydro-quinoxa-line-2,3-dione, 4-(3-phosphono-propyl)-piperazine-2-carboxylic acid (CPP), D-(E)-4-(3-phosphonoprop-2-enyl)piperazine-2-carboxylic acid (D-CPPene), SDZ-220581, PD-134705, LY-274614 and WAY-126090; quinolinic acids, such as kynurenic acid, 7-chloro-kynurenic acid, 7-chloro-thiokynurenic acid and 5,7-dichloro-kynurenic acid, prodrugs thereof, such as 4-chlorokynurenine and 3-hydroxy-kynurenine; 4-aminotetrahydrochinolin-carboxylates, such as L-689,560; 4-hydroxyquinolin-2(1H)-ones, such as L-701,324; quinoxalinediones, such as licostinel (ACEA-1021) and CGP-68,730A; 4,6-dichloro-indole-2-carboxylate derivatives such as MDL-105,519, gavestinel (GV-150,526) and GV-196,771A; tricyclic compounds, such as ZD-9,379 and MRZ-2/576, (+)-HA-966, morphinan derivatives such as dextromethorphan and dextrophan; benzomorphans, such as BIII-277CL; other opioids, such as dextropropoxyphene, ketobemidone, dextromethadone and D-morphine; amino-adamantanes, such as amantadine and memantine; amino-alkyl-cyclohexanes, such as MRZ-2/579; ifenprodil and ifenprodile-like compounds such as eliprodil and PD-196, 860; iminopyrimidines; or other NMDA-antagonists such as nitroprusside, 1-aminocyclopropane-carboxylic acid, dizocilpine (MK 801) and its analogs, (R)-ketamine, (5)-ketamine, remacemide and its des-glycinyl-metabolite FPL-12,495, AR-R-15,896, methadone, sulfazocine, AN19/AVex-144, AN2/AVex-73, Besonprodil, CGX-1007, EAB-318, and NPS-1407.

### Ketamine

Ketamine was first synthesized in the early 1960s for use as a dissociative anesthetic. It was subsequently shown to induce its effects by blocking neurotransmission at NMDAR (e.g. Javitt & Zukin, Am J Psychiatry, 148:1301-8, 1991).

There are two enantiomeric forms of ketamine: S-ketamine (esketamine) and R-ketamine (arketamine). S-ketamine is known to be approximately 4-fold more potent as an NMDAR antagonist than R-ketamine (e.g. Oye et al., J Pharmacol exp Ther 260, 1209-1213, 1992).

Ketamine is widely used in anesthesia at doses of 1-4.5 mg IV. Potential other routes of administration include oral administration or parenteral administration via transmucosal, intramuscular, subcutaneous, intranasal, transpulmonary (intra-alveolar), intraperitoneal or rectal routes of administration (e.g. Diaz et al., Paediatric Anesthesia 7:273-278, 1997; Malinovsky et al., Br J Anesthesia 77:203-7, 1996). Intranasal administration may be provided by nasal insufflation. Transpulmonary (intra-alveolar) administration may be provided by nebulization and inhalation.

At subanesthetic doses, ketamine may augment effects of other anesthetic agents. At both subanesthetic and anesthetic doses, ketamine may induce dissociative side effects including psychotomimetic effects. Both the subanesthetic and anesthetic dose effects of ketamine are related to its potency in binding to the PCP-binding site within the ion channel formed by the NMDAR and in blocking net NMDAR-mediated neurotransmission.

Esketamine (S-ketamine) is also used as a dissociative anesthetic at a dose of approximately M of that used for racemic (R,S) ketamine.

Clinical use of ketamine and esketamine is limited by their liability to induce psychosis-like (psychotomimetic) effects at anesthetic and subanesthetic doses.

### Compositions for Treatment of NMDAR Responsive Neuropsychiatric Conditions

The compositions described herein are used in treatments of NMDAR responsive neuropsychiatric conditions. The described methods include administration to a subject in need thereof an effective amount of a first composition that includes an NMDAR antagonist selected from ketamine, (S)-ketamine or (R)-ketamine and an effective amount of a second composition that is an antidepression agent. As further described herein, the NMDAR antagonist and the antidepression agent can be administered in the same pharmaceutical formulation or in separate pharmaceutical formulations. When administered in separate formulations, the MDAR antagonist and the antidepression agent can be administered at the same time or in sequence. For example, in certain embodiments, the antidepression agent can be administered to a subject first for a period of days to years, in order to stabilize the particular condition to be treated, and the ketamine agent can be administered subsequent to patient stabilization. Illustrative NMDAR antagonist-responsive conditions are described as follows.

NMDARs mediate a process termed long-term potentiation that may be excessive in disorders such as depression, obsessive compulsive personality disorder (OCD), post-traumatic stress disorder (PTSD) or other stress-related disorders (SD) and anxiety disorders such as adjustment disorders with depressive, anxious or mixed features. NMDAR excess disorders may be identified by objective biological markers such as reduced brain glutamate+glutamate (Glx) levels as detected using magnetic resonance spectroscopy (MRS) (e.g. Milak et al., Mol Psychiatry. 2016; 21(3):320-7; Kantrowitz et al. Am J Psychiatry. 2016; 173(12):1241-2).

NMDARs have also been implicated in the maintenance of neuropathic pain, especially within central regions. For example, in prior studies, acute intraperitoneal administration of the NMDAR antagonist ketamine has been shown to block only the acute stages of formalin-induced pain following systemic administration, whereas it selectively blocks the late phase of the response following intrathecal administration (e.g. Bulutcu et al., Life Sci. 71:841-53, 2002). Other NMDAR antagonists that may have a ketamine-like effect include dextromethorphan, D-methadone, and phencyclidine.

Specific pain syndromes that may respond to NMDAR antagonists such as ketamine include post-operative pain, chronic and neuropathic pain, cancer-related pain, headache, complex regional pain syndrome, fibromyalgia, and phantom-limb pain (Riccardi et al., J Clin Med 2023; 12:3256; Faisco et al., Cureus, 2024; 16:e53365).

As noted, the individual pharmaceutical compounds or pharmaceutical compositions or formulations thereof that make up each of the components of the described combination composition may be administered to the patient separately, for example in different pills or capsules, with the timing meant to ensure overlap of the components within the blood stream or other body compartments of the individual. In some embodiments, the antidepression agent is administered in accordance with FDA approved.

In some embodiments, the timing of the onset of the administration of the medications may differ. For example, in some embodiments, individuals may be receiving daily or intermittent doses of an antidepression agent for periods of 1 day up to several years, with a preferred duration of approximately 2 weeks to 6 months. Examples would include without limitation, 2 weeks, 4 weeks, 6 weeks or 8 weeks, or 1 month, 2 months, 3 months, 4 months, 5 months or 6 months.

In other embodiments, the NMDAR antagonist, and particularly ketamine or enantiomers thereof, may be administered at a daily or intermittent doses for period of 1 day up to several years, and the anti-depression agent is administered subsequently.

The compositions described herein can be administered to a subject by any route known to the art to be suitable for providing an NMDAR antagonist and an antidepression agent to a subject. It will be appreciated that if provided separately, the ketamine agent can be formulated for one type of administration (e.g. oral, intravenous), whereas the antidepression agent can be formulated the same or a different type of administration.

In some embodiments, the composition or compositions to be provided to the subject are provided orally as solid composition(s) or liquid composition(s).

Solid compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, lipids, alginic acid, or ingredients for controlled slow release. Disintegrators that can be used include, without limitation, micro-crystalline cellulose, corn starch, sodium starch glycolate and alginic acid. Tablet binders that may be used include, without limitation, acacia, methylcellulose, sodium carboxymethylcellulose, polyvinylpyrrolidone (Povidone), hydroxypropyl methylcellulose, sucrose, starch, and ethylcellulose.

In some embodiments, liquid compositions for oral administration prepared in water or other aqueous vehicles can include solutions, emulsions, syrups, and elixirs containing, together with the active compound(s), wetting agents, sweeteners, coloring agents, and flavoring agents. Various liquid and powder compositions can be prepared by conventional methods for inhalation into the lungs of the patient to be treated.

In some embodiments, the first therapeutic agent (i.e., the NMDAR antagonist) or the second therapeutic agent (i.e., the antidepression agent) may be formulated as an injectable composition, which may contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like).

In some embodiments, the first therapeutic agent or the second therapeutic agent may be formulated as an intravenous injection, the compounds may be administered by the drip method, whereby a pharmaceutical composition containing the active compound(s) and a physiologically acceptable excipient is infused. In some embodiments, the infusion rates would be dynamically modulated to maximize therapeutic effects while minimizing side effects. Physiologically acceptable excipients for use in formulations of the present disclosure may include, for example, 5% dextrose, to 0.9% saline, Ringer's solution or other suitable excipients. For intramuscular preparations, a sterile composition of a suitable soluble salt form of the compound can be dissolved and administered in a pharmaceutical excipient such as Water-for-Injection, 0.9% saline, or 5% glucose solution, or depot forms of the compounds (e.g., decanoate, palmitate, undecylenic, enanthate) can be dissolved in sesame oil. Alternatively, the pharmaceutical composition can be formulated as a chewing gum, lollipop, or the like. In other embodiments, the formulations as herein described, in particular with regard to oral formulations, are envisioned to comprise slow-release tablet formulations. Such slow release tablet formulations may, for example, comprise commercially available formulations containing known anti-depressant medications, such as, for example, Effexor® or Seroquel®, both of which are already available in extended length (XR) formulations, however the formulation may be modified to further incorporate D-methadone.

In other embodiments, the formulations as herein described, in particular with regard to oral formulations, are envisioned to comprise both short acting and extended release formulations. Extended release formulations have the advantage inter alia of minimizing the difference between peak and trough levels of drug, and thereby to increase effectiveness and/or reduce side effects of a medication.

Other compounds can be used to control release include cellulose, ethylcellulose, gelatin, hypromellose, iron oxide, and titanium oxide. In some matrix systems, drug release is controlled mainly by diffusion through matrix pores and not by the erosion at the polymers. Drug delivery can also be controlled by use of reservoir type systems in which release is controlled by osmotic gradient across the coating membrane. Capsules can be manufactured which contain granules with different microencapsulation properties which can be blended to achieve a composition that has a desired release rate. In some embodiments, the invention contemplates employing/providing a sustained release formulation containing the agents as herein described, further comprising a gelling agent, preferably hydroxypropyl methylcellulose, and 11-[4-[2-(2-hydroxyethoxy)ethyl]-1-piperazinyl]dibenzo-[b.f][1,4]thiazepin e, or a pharmaceutically acceptable salt thereof, together with one or more pharmaceutically acceptable excipients. The term gelling agent as used herein means any substance, which forms a gel when in contact with water.

Methods for the formulation of the described regimens herein are well known, and the skilled artisan will appreciate that it is straightforward to prepare the oral dosage regimens as herein described. Applicants, for example, refer to Gibaldi's Drug Delivery Systems in Pharmaceutical Care, Desai A & Lee M (eds), Bethesda, Md.: American Society of Health-System Pharmacists, 2007.

It is to be understood that the described compositions are provided by “co-administration” and that the co-administration of either of the two active ingredients to a subject can, in certain embodiments, be combined in a single formulation. In other embodiments, the active ingredients are provided in separate formulations, and which administration can be coincident or staggered.

As noted, the compositions described herein can be administered by a variety of well-established medicinal routes including intravenously, intraperitoneally, parentally by nasal insufflation or pulmonary inhalation, intramuscularly, subcutaneously or orally.

In some embodiments, solid compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, lipids, alginic acid, or ingredients for controlled slow release. Disintegrators that can be used include, without limitation, micro-crystalline cellulose, corn starch, sodium starch glycolate, and alginic acid. Tablet binders that may be used include without limitation, acacia, methylcellulose, sodium carboxymethylcellulose, polyvinylpyrrolidone (Povidone), hydroxypropyl methylcellulaose, sucrose, starch, and ethylcellulose.

In some embodiments, the subject suffers from schizophrenia, or a depressive disorder, including major depressive disorder or bipolar depressive disorder.

In some embodiments, the invention provides a method for reducing the incidence of suicide or reducing the severity of suicide ideation and behaviors in a subject or population in need thereof, by providing the subject with an oral or parenteral or parenteral dosage regimen of a composition as herein described.

It is expected that a subject undergoing treatment with the methods and compositions described herein will experience significant improvements in depression. Relative to subjects treated with alternative treatments for depression, subjects treated accordingly will experience, in some embodiments, greater improvement, or more long-lasting improvement, as measured by any clinically recognized assessment method for depression (e.g., the 21-item Hamilton Depression Rating Scale). Similarly, a subject undergoing treatment with the described compositions and methods can experience significant improvements in anxiety. Relative to subjects treated with alternative treatments for anxiety, subjects treated accordingly will experience, in some embodiments, greater improvement, or more long-lasting improvement, as measured by any clinically recognized assessment method for anxiety (e.g., the Hamilton Anxiety Rating Scale). Likewise, a subject undergoing treatment with the described compositions and methods can experience significant improvements in akathisia.

Relative to subjects treated with alternative treatments for akathisia, subjects treated accordingly will experience, in some embodiments, greater improvement, or more long-lasting improvement, as measured by any clinically recognized assessment method for akathisia (e.g., the Barnes Akathisia Rating Scale). Also, a subject undergoing treatment with the described compositions and methods can experience significant improvements in psychosis. Relative to subjects treated with alternative treatments for psychosis, subjects treated accordingly will experience, in some embodiments, greater improvement, or more long-lasting improvement, as measured by any clinically recognized assessment method for psychosis (e.g., the Positive and Negative Symptom Scale).

The following examples describe certain embodiments of the invention and should not be construed as limiting the scope of what is encompassed by the invention in any way.

### EXAMPLES

### Example 1: Effect of Combined Ketamine and Antidepressants in the Rodent Amphetamine-Induced Locomotor Activity Assay (LMA)

We have previously demonstrated that anti-depressants unexpectedly reverse the psychotomimetic liability of D-cycloserine as reflected in amphetamine-induced locomotor activity in rodents while not inhibiting its antidepression effects, as reflected in immobility time during the forced swim assay. Here, we evaluated similar interactive effects involving ketamine, S-ketamine and antidepressants.

### Amphetamine-Induced Locomotor Activity.

Amphetamine-induced locomotor activity (LMA) in an open-field test is used as a measure of liability to induce a psychosis-like effect. In general, agents that induce locomotor activity in this assay either alone or in combination with amphetamine are considered to have significant liability to induce psychosis-like effects in humans. NMDAR antagonists, such as D-cycloserine (US Patent Publication No. US 2022/0143041) or ketamine (Lai et al., Neurochem Res 39:2180-2188, 2014) are known to increase locomotor activity on their own and enhance the effects of amphetamine.

Antipsychotic agents are known to reduce amphetamine-induced locomotor activity. In general, anti-depressants are not thought to affect amphetamine-induced locomotor hyperactivity. However, we have previously shown that specific antidepressant medications may reverse effects of D-cycloserine (US Patent Publication No. US 2022/0143041), suggesting specific beneficial effects in combination with NMDAR antagonists.

Here we investigated the effects of antidepression agents including SNRI, SSRI, TeCA and SDRI agents in the amphetamine-induced locomotor activity in both the absence and presence ketamine.

Testing was performed at Psychogenics, Inc. located in Paramus, NJ. The open field chambers are Plexiglas square chambers (27.3×27.3×20.3 cm; Med Associates Incs., St Albans, VT) surrounded by infrared photobeam sources (16×16×16). Horizontal activity (distance traveled) and vertical activity (rearing) are measured from consecutive beam breaks. On the day of testing, animals were brought to the experimental room for at least 1-hour acclimation prior to start of treatment. Animals were administered with vehicle or test articles and placed in the OF. Baseline activity was recorded for 30 min, following which mice received either Amphetamine (4 mg/kg) or saline and were placed back in the OF chambers for a 60-minute session.

The primary dependent measures for statistical analyses were average open field activity during the 0-30 min (pre-amphetamine baseline) versus activity during 30-90 min (post-amphetamine administration). Analyses were conducted using univariate ANOVA and repeated-measures ANOVA across the pre- and post-amphetamine intervals. Amphetamine-effects were analyzed using within-subject statistics. The effects of ketamine and antidepression agents alone and in combination were assessed using between-subject statistics. Post-hoc tests were performed using LSD The preset level for significance was p<0.05. For all studies described in this application, data from mice that died or become incapacitated during the course of the study were disregarded for statistical analyses.

Duloxetine (20 mg/kg or 40 mg/kg), citalopram (10 mg/kg), sertraline (20 mg/kg), venlafaxine (40 mg/kg), mirtazapine (10 mg/kg), milnacipran (40 mg/kg), fluoxetine (10 mg/kg) and bupropion (10 mg/kg) was dissolved in PTS vehicle (5% PEG 200:5% Tween 80:90% saline) and administered IP at a dose volume of 10 ml/kg 30 min prior to Amphetamine.

S-ketamine (10 mg/kg) and ketamine (10 mg/kg or 25 mg/kg) were administered as a cocktail with duloxetine (20 mg/kg or 40 mg/kg), citalopram (10 mg/kg), sertraline (20 mg/kg), venlafaxine (40 mg/kg), mirtazapine (10 mg/kg), milnacipran (40 mg/kg), fluoxetine (10 mg/kg) or bupropion (10 mg/kg) at a dose volume of 10 ml/kg 30 min prior to Amphetamine

Amphetamine (4 mg/kg) was dissolved in saline and injected IP at a dose volume of 10 ml/kg.

For statistical purposes, the effects of antidepressants were first analyzed by collapsing across the SNRI, SSRI, TeCA and NDRI groups. Follow-up analyses then evaluated the relative effects of the different groups, followed by assessment of each individual compound.

Across all antidepression treatments, ketamine treatment significantly increased locomotor activity (F[1,432]=25.3, p<0.001) in the pre-amphetamine baseline. Furthermore, when effects of the compounds were evaluated individually, all three ketamine formulations significantly increased activity versus the no ketamine condition (Table 1).

TABLE 1
Effects of ketamine on locomotor activity in the pre-amphetamine
baseline. Values are estimated marginal means and SEM.

| Condition | Mean | SEM | p vs. No Ket |
|---|---|---|---|
| No Ketamine | 816.1 | 45.9 | — |
| Ketamine 10 mg/kg | 929.3 | 45.9 | <.001 |
| Ketamine 25 mg/kg | 1004.2 | 45.9 | <.001 |
| S-ketamine 10 mg/kg | 1264.5 | 45.9 | .003 |

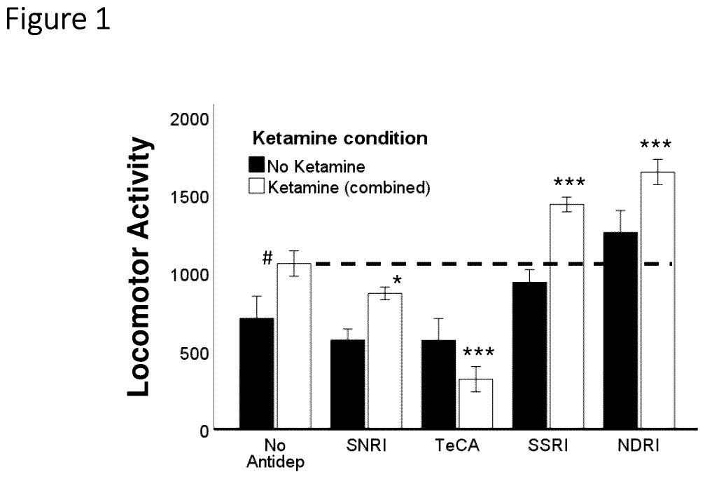

In the pre-amphetamine baseline, anti-depressants showed differential effects on ketamine-induced potentiation of locomotion by class, as reflected in a highly significant main effect of antidepressant-type (F[4,430]=34.1, p<0.001) and a highly significant ketamine X antidepressant-type interaction (F4,430=4.08, p=0.003) (FIG. 1).

In post-hoc tests, SNRI (p=0.003) and TeCA (p<0.001) significantly decreased locomotor activity relative to vehicle, whereas SSRI (p<0.001) and SDRI (p<0.001) significantly increased locomotor activity.

When SNRI were evaluated by individual compound, significant individual effects (decreases) were observed for duloxetine 40 mg/kg (p<0.001) and milnacipran 40 mg/kg (p<0.001) across ketamine formulations (Table 2).

TABLE 2
Effects of antidepressants on locomotor activity in the pre-amphetamine baseline
in combination with ketamine. Values are estimated marginal means and SEM.

| | | | | | | | | | p vs. No |
|---|---|---|---|---|---|---|---|---|---|
| | | | Ketamine | | Ketamine | | Esketamine | | Anti [symbol] ep |
| | No Ketamine | | 10 mg/kg | | 25 mg/kg | | 10 mg/kg | | (across ke [symbol] |
| Condition | mean | sem | mean | sem | mean | sem | mean | sem | doses) |
| No Anti [symbol] ep | 710.4 | 133.0 | 9 [symbol] 1.8 | 133.0 | 1358.8 | 133.0 | 859.8 | 133.0 | — |
| SNRI (comb) | 571.2 | [symbol] .5 | 82 [symbol] .8 | 66.5 | 10 [symbol] 5.1 | [symbol] .5 | 718.5 | [symbol] .5 | 0.017 |
| Du [symbol] oxetine 20 mg/kg | 687.5 | 101.1 | 828.2 | 101.1 | 1186.0 | 101.1 | 731.5 | 101.1 | NS |
| Du [symbol] oxetine 40 mg/kg | 473.7 | 101.1 | 623. [symbol] | 101.1 | 1025.8 | 101.1 | [symbol] 41.8 | 101.1 | <.001 |
| Venlafaxine 40 mg/kg | 5 [symbol] 2.8 | 101.1 | 1143. [symbol] | 101.1 | 1399. [symbol] | 101.1 | 1031.9 | 101.1 | NS |
| M [symbol] acipran 40 mg/kg | 5 [symbol] 0.7 | 101.1 | [symbol] 99.9 | 101.1 | 648.9 | 101.1 | 469.0 | 101.1 | <.001 |
| Mirtrzapine 10 mg/kg | 569.1 | 133.0 | 531. [symbol] | 133.0 | 22 [symbol] .4 | 133.0 | 204.5 | 133.0 | <.001 |
| S SRI (comb) | 940. [symbol] | 7 [symbol] .8 | 1274.4 | 7 [symbol] .8 | 17 [symbol] 1.5 | 7 [symbol] .8 | 17 [symbol] 1.5 | 7 [symbol] .8 | <.001 |
| Ci [symbol] alap [symbol] 10 mg/kg | [symbol] 42.8 | 141.4 | 1445. [symbol] | 141.4 | 2022.2 | 141.4 | 1405.0 | 141.4 | <.001 |
| Sertraline 20 mg/kg | 1135.0 | 141.4 | 1478.2 | 141.4 | 1788.7 | 141.4 | 1 [symbol] 13.1 | 141.4 | <.001 |
| Fluoxe [symbol] ine 10 mg/kg | 844.1 | 141.4 | 899. [symbol] | 141.4 | 1473.5 | 141.4 | [symbol] 24.0 | 141.4 | NS |
| Bupropion 10 mg/kg | 1259.1 | 133.0 | 1515.1 | 133.0 | 1 [symbol] .9 | 133.0 | 1555.0 | 133.0 | <.001 |

[symbol]

indicates data missing or illegible when filed

The effects of duloxetine were independently significant in the presence of S-ketamine 10 mg/kg (p=0.05), ketamine 10 mg/kg (p=0.044) and ketamine 25 mg/kg (p=0.003). The effects of milnacipan were independently significant for S-ketamine (p<0.001) and ketamine 25 mg/kg (p<0.001).

The effects of mirtazapine 10 mg/kg were independently significant in the presence of ketamine 10 mg/kg (p=0.002), ketamine 25 mg/kg (p<0.001), and S-ketamine (p<0.001),

When SSRI were evaluated by individual compound, significant independent effects (increases) were observed for citalopram 10 mg/kg and sertraline 20 mg/kg.

The effects of citalopram were independently significant in combination with S-ketamine 10 mg/kg (p=0.002), ketamine 10 mg/kg (p<0.001) and ketamine 25 mg/kg (p<0.001). The effects of sertraline were independently significant in the presence of esketamine (p<0.001), ketamine 10 mg/kg (p<0.001) and ketamine 25 mg/kg (p=0.02).

The effects of bupropion were independently significant in the presence of ketamine 10 mg/kg (p<0.001), ketamine 25 mg/kg (p<0.001) and S-ketamine (p<0.001).

The effects of SNRI (p<0.001) and TeCA (p<0.001) were also significantly different from those of SSRI across ketamine formulations.

Following amphetamine administration, the main effects of ketamine (F[1,390]=15.7, p<0.001), antidepressant type (F[4,390]=10.2, p<0.001) and the ketamine X antidepressant-type interaction (F4,390=2.46, p=0.045) were again significant.

SSRI as a group (p=0.045) and mirtazapine (p=0.002) again reduced locomotor activity relative to vehicle. SSRI showed no significant effects. In this case, bupropion significantly decreased activity (p<0.001), as opposed to the increase observed pre-amphetamine.

When SNRI were evaluated by individual compound, significant individual effects (decreases) were observed for duloxetine 20 mg/kg (p<0.001), venlafaxine 40 mg/kg (p=0.006) and milnacipran 40 mg/kg (p<0.001) across ketamine formulations.

The effects of duloxetine 20 mg/kg were independently significant in the presence of S-ketamine (p=0.049) and ketamine 25 mg/kg (p=0.042). The effects of venlafaxine were independently significant in the presence of esketamine (p=0.037) and ketamine 25 mg/kg (p=0.008). The effects of milnacipran were independently significant in the presence of esketamine (p=0.011), ketamine 10 mg/kg (p=0.002) and ketamine 24 mg/kg (p<0.001).

When SSRI were evaluated by individual compound, a significant individual effect (decrease) was observed for citalopram (p<0.001) across ketamine formulations.

The effect of citalopram was independently significant in the presence of S-ketamine (p=0.012), ketamine 10 mg/kg (p=0.009) and ketamine 25 mg/kg (p=0.029).

The effects of bupropion were independently significant in the presence of S-ketamine (p<0.001) and ketamine 25 mg/kg (p=0.003).

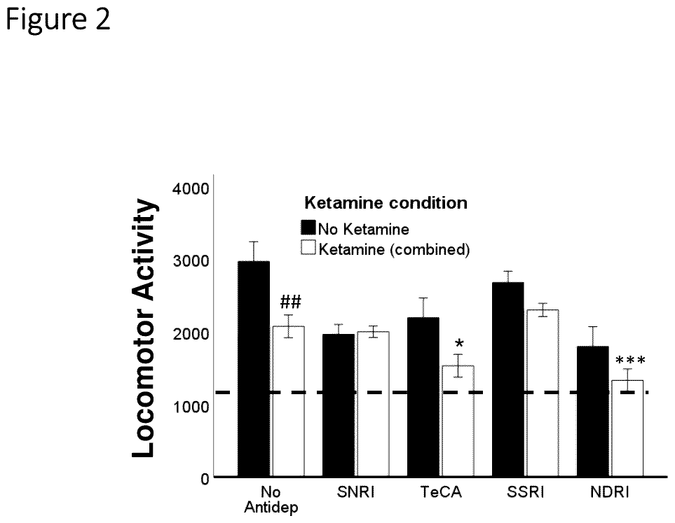

In the post-amphetamine period, there was a significant main effect of ketamine treatment across ketamine formulations (F[1,390]=15.7, p<0.001), reflecting a reduction in locomotor activity related to the no-ketamine conditions (FIG. 2).

There was also a significant main effect of antidepression treatment across all antidepressant types (F[4,390]=10.2, p<0.001) and a significant ketamine X antidepressant type (F[4,390=2.46, p=0.045), reflecting significant effects of SNRI (p<0.045), TeCA (p=0.002), and NDRI (p<0.001) relative to no antidepressant across ketamine conditions. The effects of SSRI were not significant versus the no-antidepressant treatment (p=0.5), but were differential versus other antidepressant types (all p<0.01).

Least locomotor activity in the post-amphetamine period occurred in the presence of bupropion (NDRI). Average locomotor activity following amphetamine administration in the presence of combined ketamine and NDRI was not significantly different from the pre-amphetamine baseline in the absence of NMDRI (p=0.2)

Overall, these findings show an unexpected differential ability of SNRI and TeCA to inhibit locomotor activity induced by ketamine agents, indicating likely antipsychotic effect in combination with ketamine, as well as a synergistic antidepression effect.

### Higher Dose Ketamine

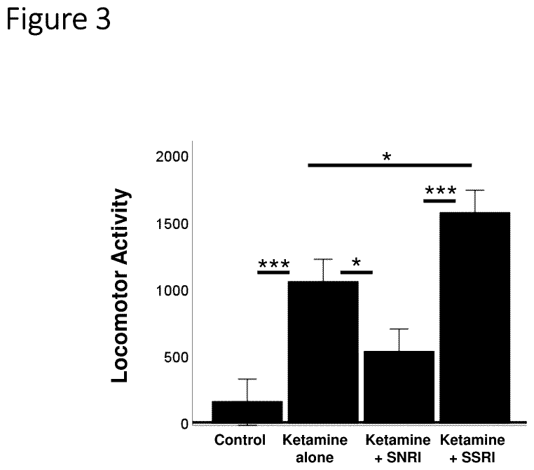

The differential effects of duloxetine and sertraline were further confirmed in combination with a higher dose of ketamine. In these experiments, sertraline 20 mg/kg and duloxetine (40 mg/kg) were administered prior to injection of ketamine 100 mg/kg (FIG. 3).

As expected, the main effect of treatment condition was highly significant (F[3,36]=12.6, p<0.001). In post-hoc tests, the difference between ketamine alone and control (vehicle) was strongly significant (p<0.001), reflecting a ketamine-induced increase in locomotion.

The difference between ketamine alone and ketamine+duloxetine was also strongly significant (p<0.001), reflecting a reduction in locomotor activity induced by duloxetine in combination with ketamine. Moreover, locomotor activity in the presence of combined ketamine+duloxetine was not significantly different from control conditions (p=0.13), indicating a reversal of ketamine-induced psychotomimetic-like effects.

Locomotor activity in the presence of combined ketamine+sertraline was significantly greater than that observed in the presence of ketamine alone (p=0.042) or combined ketamine+duloxetine (p<0.001), indicating a significant differential effect of duloxetine and sertraline.

When animals were administered repeated ketamine (50 mg/kg) for 6 days, both the increase in locomotor activity versus baseline (p<0.001) and the additional increase in locomotor activity with combined ketamine+sertraline versus ketamine alone (p<0.001) remained strongly significant.

Overall, these are the first studies to demonstrate the ability of specific antidepressant types, including SNRI and TeCA to inhibit ketamine-induced increases in locomotor activity in both the absence and presence of amphetamine. In addition, these are the first studies to demonstrate differential effects of different antidepressant classes.

These studies show unique beneficial effects of SNRI and TeCA (mirtazapine) in combination with ketamine agents.

In addition, we demonstrate differential effects between SNRI and SDRI on pre-versus post-amphetamine effects, indicating likely differential utility for differential clinical populations

No significant effects of ketamine or antidepressants were observed on rearing behaviors, arguing against general effects on motor coordination.

### Rodent Elevated Plus Maze (EPM)

The EPM serves as an index of anxiety/akathisia, such that decreased open arm entries represent an increased anxiety state and increased akathisia, whereas increased open arm entries reflect an anti-anxiety and anti-akathisia effect.

EPM assessments were performed at Pharmaseed, Israel. The EPM assessments were conducted on Day 1 as baseline, and on Day 8 30 minutes post administration, according to Pharmaseed's SOP No. 120 “Elevated Plus Maze Test for Mice”. Animals were acclimated to the testing room for at least 30 minutes prior to the testing then placed in the EPM apparatus for ten minutes. Animals were recorded using Ethovision tracking system. Statistical comparisons were performed using Univariate analysis of variance (ANOVA) with indicated factors. Post-hoc tests were performed using least significant difference (LSD) testing. The cutoff for significance was p<0.05.

All drugs were administered i.p. Antidepressants included sertraline (SSRI) 20 mg/kg; citalopram (SSRI) 10 mg/kg; duloxetine (SNRI) 40 mg/kg; Venlafaxine (SNRI) 80 mg/kg. S-Ketamine was administered at a dose of 10 mg/kg.

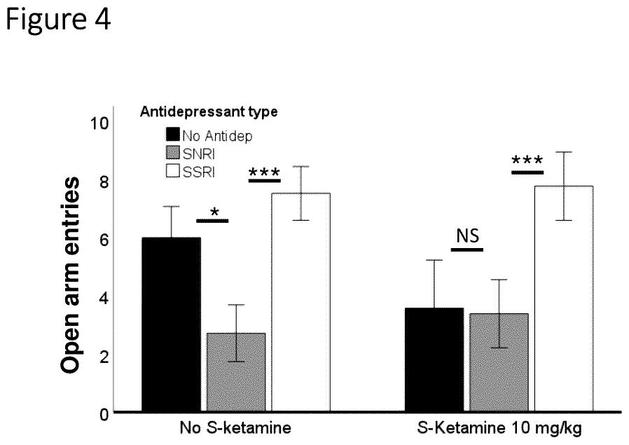

Across all conditions, there was a significant main effect of anti-depressant type (F[2,132]=133, p<0.001) that remained significant following covariation for closed arm entries (F[2,131]=13.3, p<0.001), such that SNRI significantly decreased open arm entries in the absence of S-ketamine (p=0.03) whereas effects of SSRI were not significant. The differential effect of SNRI and SSRI was highly significant (p<0.001). (FIG. 4)

The effects of S-ketamine were not statistically significant overall (F[1,131]=0.3, p=0.6) or in any antidepressant condition independently.

However, in the presence of S-ketamine the reduction in open arm entries by SNRI was no longer significant relative to S-ketamine alone (p=0.9). The difference between SNRI and SSRI was still strongly significant (p=0.009).

When analyses were performed by individual antidepressant agent, both duloxetine (p<0.001) and venlafaxine (p<0.024) reduced open arm entries significantly relative to the no antidepressant condition in the absence of S-ketamine. In the presence of S-ketamine these differences were no longer significant.

These data demonstrate a differential liability of SNRI and SSRI to induce anxiety/akathisia-type symptoms, and the unexpected ability of S-ketamine to mitigate the behavioral changes induced by SNRI. Rodent forced swim test (FST).

The rodent forced swim test (FST) is a widely used animal model for predicting antidepression effects of pharmaceutical agents. The ability of compounds to reduce immobility time in the FST predicts their likelihood of inducing clinically significant antidepression effects in humans.

FST testing was performed at Pharmaseed Ltd., Ness Ziona, Israel. Male BALB/cOlaHsd mice were obtained from Envigo and were 10-11 weeks at study initiation. Animals were housed under standard laboratory conditions. Test agents including S-ketamine and antidepression agents were administered by intraperitoneal (IP) administration.

On the day of testing, animals were acclimated to the testing room for at least 15 minutes prior to the administration of test compounds. The FST was conducted 30 minutes following administration of the test compounds. Mice were then placed for six minutes in a cylindrical Plexiglas container (around 20 cm diameter) filled with water at 22-25° C. and a depth of at least 20 cm. The tests were recorded using a video camera and analyzed offline. Behavioral monitoring was performed from 2 to 6 minutes after placing the mouse in the water.

The main outcome measure used for statistical analysis is the cumulative inactive duration (time) during the FST. Statistical comparisons were performed using Univariate analysis of variance (ANOVA) with indicated factors. Post-hoc tests were performed using least significant difference (LSD) testing. The cutoff for significance was p<0.05.

Antidepression compounds were divided into SNRI and SSRI. The following compounds were tested in this study, alone and in combination:
S-ketamine (NMDAR antagonist), which was administered at a dose of 10 mg/kg.
Duloxetine (SNRI), which was administered at a dose of 40 mg/kg
Venlafaxine (SNRI), which was administered at a dose of 40 mg/kg
Citalopram (SSRI), which was administered at a dose of 10 mg/kg
Sertraline (SSRI), which was administered at a dose of 20 mg/kg

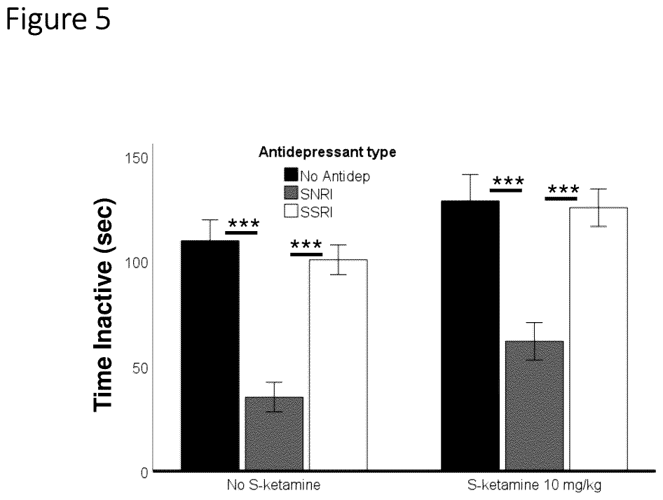

The primary dependent variable was time inactive during the FST (FIG. 5).

There was a significant main effect of anti-depressant type (F[2,124]=41.1, p<0.001), reflecting significantly reduced inactivity time in the presence of anti-depressants (F1,126=11.9, p<0.001) than in their absence.

In the absence of S-ketamine, post-hoc tests showed a significant reduction in time inactive during the FST relative to both vehicle control (p<0.001) and SSRI (p<0.001).

There was also a significant main effect of S-ketamine (F[1,124=9.50, p=0.003), reflecting greater time inactive in the presence versus absence of S-ketamine across anti-depressant conditions.

Nevertheless, SNRI showed continued anti-depressant effects in the presence of S-ketamine, as reflected in reduced inactivity time in the presence of combined S-ketamine+SNRI versus in the presence of S-ketamine alone (p<0.001).

When tested independently, both duloxetine (p<0.001) and venlafaxine (p<0.001) induced significant reductions in inactivity time in the presence of S-ketamine when compared with S-ketamine alone.

These findings demonstrate that the anti-depressant effects of SNRI remain significant when the agents are combined with S-ketamine, reflecting significant beneficial effects of this combination.

### SUMMARY

Overall, NMDAR antagonists such as D-cycloserine have known anti-depressant properties in humans, but their clinical effects are limited by the propensity to induce psychotomimetic side effects, which can be modeled using the rodent locomotor assay. Here, as expected ketamine compounds, induced significant locomotor activity in the absence of amphetamine, consistent with their known psychotomimetic side effects. These effects were unexpectedly prevented by simultaneous treatment with SNRI-, TeCA-, and NDRI-type antidepressants, reflecting distinct utility of the combination of NMDAR antagonists and these types of antidepressants.

The combination of S-ketamine+SNRI also showed synergistic effects in the rodent depression (FST) and anxiety/akathisia (EPM) models, showing unexpected benefits of this combination

Doses may be scaled between mice and humans using a scaling factor of 0.081. and a dose range of 10× above or below the mean dose (e.g. Shen et al., Clin Transl Sci 12:6-19, 2019). Thus, the present data would suggest an optimal dose in the range of 0.08-8 mg/kg for ketamine formulations. However, other dose ranges are possible as well, for example 0.01-0.1 mg/kg, 0.1-0.5 mg/kg; 0.5-1.0 mg/kg; 1.0-5.0 mg/kg; 5.0-10.0 mg/kg; 10.0-20.0 mg/kg; 20.0-50.0 mg/kg. Other factors, such as age, sex, ethnicity and medical conditions may also determine optimal clinical doses.

Similarly, based upon pharmacokinetic scaling, appropriate doses of duloxetine and venlafaxine will be in range of 0.32-32 mg/kg. Appropriate doses of mirtazapine and bupropion would be in the range of 0.08-8.0 mg/kg.

As reported previously (e.g. U.S. Pat. No. 11,013,721B2), a preferred use of the combination treatment is selection of individuals who are poorly responsive to treatment with antidepressant drugs and have been receiving a stable therapeutic dose of an approved antidepressant drug for at least 8 weeks before study entry. For example, such patients may met criteria for refractory depression, defined as a >20 score on the 21-item HAM-D, despite at least 2 prior adequate antidepressant medications (e.g. SSRIs, SNRIs, TCAs) trials during the current depression episode.

For such individuals, the ketamine agent would be added to the ongoing stable therapeutic dose at a dose sufficient to block NMDAR. Treatment may be administered either as a single acute treatment to provide rapid improvement in symptoms after which individuals are transitioned to an alternative, orally active NMDAR antagonist including without limitation D-cycloserine, D-methadone, or dextromethorphan; or as repeated treatments.

For acute treatment, preferred routes of administration include intravenous, transpulmonary/intra-alveolar by inhalation, transmucosally, intranasally by insufflation, intramuscularly or transcutaenously.

For repeated administration, potential frequencies for repeat administration include daily, weekly, or monthly. Intermediate rates of administration are also possible including 6 times per week, 5 times per week, 4 times per week, 3 times per week or 2 times per week, or between 1- and 30-times per month. Multiple doses may also be delivered in a single day. For example, 2 doses per day or 3 doses per day.

Preferred antidepressants include mirtazapine, duloxetine, venlafaxine and bupropion.

In addition, preferred individuals for treatment with combined NMDAR antagonist and antidepression agent may be experiencing suicidal ideation or guilt feelings, which may be a consequence either of the illness or the antidepressant treatment. In such cases, an additional role of the NMDAR antagonist is to reduce guilt feelings and associated suicidal ideation. Suicidal ideation may be related to akathisia, which may be reduced by simultaneous administration of an NMDAR antagonist treatment.

All references cited herein, including U.S. patents and published patent applications, international patents and patent applications, and journal references or other publicly available documents, are incorporated herein by reference in their entireties to the same extent as if each reference had been specifically cited for the portion or portions of such reference applicable to the section of this application in which it is cited or to which it is relevant.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as my invention all that comes within the scope and spirit of these claims.

1. A pharmaceutical composition for treatment of an NMDAR antagonist-responsive disorder, consisting essentially of two pharmaceutically active agents, wherein the first pharmaceutically active agent is a ketamine agent, and the second pharmaceutically active agent is an antidepression agent.

2. The pharmaceutical composition of claim 1, wherein the ketamine agent is racemic (R,S) ketamine, S-ketamine (esketamine), or R-ketamine (arketamine), and wherein the ketamine agent is formulated for provision at an acutely or intermittent dose of 0.08-8.0 mg/kg.

3. The pharmaceutical composition of claim 2, wherein the ketamine and antidepression agents are formulated for oral administration.

4. The pharmaceutical composition of claim 2, wherein the ketamine agent is formulated for parenteral administration, and the antidepression agent is formulated for oral administration.

5. The pharmaceutical composition of claim 1, wherein the ketamine agent and antidepression agent are both formulated for parenteral administration.

6. The pharmaceutical compositions of claim 5, wherein the parenteral administration is intravenous, intramuscular, subcutaneous, transmucosal, intranasal, transpulmonary, intraperitoneal or rectal administration.

7. The pharmaceutical composition of claim 1, wherein the antidepression agent is an SNRI, SSRI, TeCA, NDRI, or atypical antipsychotic.

8. The pharmaceutical composition of claim 4, wherein the antidepression agent is duloxetine formulated at a dose of 0.32-32 mg/kg, venlafaxine formulated at a dose of 0.32-32 mg/kg, mirtazapine formulated at a dose of 0.08-8 mg/kg or bupropion formulated at a dose of 0.08-8.0 mg/kg.

9. The pharmaceutical composition of claim 1, wherein the NMDAR responsive disorder is selected from the group consisting of major depression, bipolar depression, OCD, PTSD, and pain syndromes.

10. A method for treatment of an NMDAR antagonist-responsive disorder comprising: administering to a subject in need thereof therapeutically effective amounts of a first compound and a second compound, wherein the first compound is a ketamine agent and the second is an antidepression agent comprising an antidepressant or an atypical antipsychotic.

11. The method of claim 10, wherein the antidepressant is an SNRI, SSRI, TeCA, NDRI or atypical antipsychotic.

12. The method of claim 11, wherein the antidepression agent is duloxetine formulated at a dose of 0.32-32 mg/kg, venlafaxine formulated at a dose of 0.32-32 mg/kg, mirtazapine formulated at a dose of 0.08-8 mg/kg or bupropion formulated at a dose of 0.08-8.0 mg/kg.

13. The method of claim 10, wherein the NMDAR antagonist-responsive disorder is selected from the group consisting of major depression, bipolar depression, OCD, PTSD, and pain syndromes.

14. The method of claim 13, wherein the NMDAR antagonist-responsive disorder is major depression.

15. The method of claim 10, wherein the ketamine and antidepression agents are both administered orally.

16. The method of claim 10, wherein the ketamine agent is administered parenterally and the antidepression agent is administered orally.

17. The method of claim 10, wherein the ketamine and antidepression agents are both administered parenterally.

18. The method of claim 17, wherein the parenteral administration is intravenous, intramuscular, subcutaneous, transmucosal, intranasal, transpulmonary, intraperitoneal or rectal administration.

19. A method for the treatment of refractory major depressive disorder in a subject, comprising:
administering to the subject an antidepression agent selected from the group consisting of duloxetine, venlafaxine, mirtazapine, and bupropion, used in accordance with FDA approved (“package insert”) treatment guidelines for a period of at least 8 weeks, and
if the subject is determined to have refractory depression based upon clinical rating, administering to the subject a ketamine agent in addition to the antidepression agent, wherein the ketamine agent is administered parenterally.

20. The method of claim 19, wherein the ketamine agent is racemic (R,S) ketamine, (S)-ketamine, or (R)-ketamine.
